(19) United States
(12) Patent Application Publication
(10) Pub. No.: US 2025/0327732 A1
(43) Pub. Date: Oct. 23, 2025

(54) METHODS FOR EXTRACTING WEAR PARTICLE FEATURE SIGNALS BASED ON SEGMENTATION ENTROPY

(71) Applicant: CHONGQING UNIVERSITY OF POSTS AND TELECOMMUNICATIONS, Chongqing (CN)

(72) Inventors: Jiufei LUO, Chongqing (CN); Wei LIU, Chongqing (CN); Song FENG, Chongqing (CN); Sheng LU, Chongqing (CN); Dan JIANG, Chongqing (CN); Haiqing LI, Chongqing (CN); Hongzheng SONG, Chongqing (CN); Xin LIU, Chongqing (CN); Ziqiang ZHANG, Chongqing (CN)

(73) Assignee: CHONGQING UNIVERSITY OF POSTS AND TELECOMMUNICATIONS, Chongqing (CN)

(21) Appl. No.: 19/239,921

(22) Filed: Jun. 16, 2025

Related U.S. Application Data
(63) Continuation-in-part of application No. PCT/CN2023/135863, filed on Dec. 1, 2023.

(30) Foreign Application Priority Data
Dec. 16, 2022 (CN) 202211625240.1

(51) Int. Cl. G01N 15/06; G01N 33/28

(52) U.S. Cl. CPC G01N 15/0656; G01N 33/2858; G01N 33/2888

(57) ABSTRACT

A method for extracting a wear particle feature signal based on segmentation entropy is provided, including obtaining a raw signal to be processed by performing real-time data acquisition using a lubricating oil wear particle monitoring system; obtaining a preprocessed signal by performing low-pass filtering and harmonic interference suppression on the raw signal to be processed; dividing the preprocessed signal into a plurality of time domain sequence segments with a sliding window; calculating segmentation entropy corresponding to each time domain sequence segment, normalizing a segmentation entropy set to obtain normalized segmentation entropy; obtaining an adaptive threshold through curve fitting based on empirical cumulative distribution of normalized segmentation entropy, obtaining a plurality of non-zero discrete time domain signal segments by segmenting the preprocessed signal by the adaptive threshold; and obtaining final extraction results of the wear particle feature signal by excluding residual noise interference through target signal feature recognition indices.

### CROSS-REFERENCE TO RELATED APPLICATIONS

This application is a Continuation-in-part of International Application No. PCT/CN2023/135863, filed on Dec. 1, 2023, which claims priority to Chinese Application No. 202211625240.1, filed on Dec. 16, 2022, the entire contents of each of which are incorporated herein by reference.

### TECHNICAL FIELD

The present disclosure relates to the technical field of oil wear particle monitoring, and in particular, to methods for extracting wear particle feature signals based on segmentation entropy.

### BACKGROUND

Ferromagnetic wear particles contained in the lubricating oil of mechanical equipment are important information carriers. By analyzing information of wear particle features, the wear condition of the mechanical equipment can be effectively reflected. An inductive particle detection sensor, based on the principle of inductance, has been widely used in the lubricating oil wear particle monitoring of mechanical equipment due to its high precision, fast detection speed, and independence from oil impurities. However, in practice, raw signals collected by the sensor not only contain wear particle induced voltage signals, but also a large amount of interfering noise, which pose a serious challenge to the identification and extraction of the wear particle induced voltage signals. To improve the detection capability of the inductive particle detection sensor, researchers have done a great deal of work in structural optimization. Nevertheless, after a structure of the sensor is fixed, the location where the sensor is mounted is still affected by noise, electrical impulses, and harmonic interferences, and the accurate identification and counting of the ferromagnetic wear particles are still difficult tasks.

Much literature has employed methods for processing signals to improve detection performance of the sensor. The methods are mainly categorized into a decomposition class and a non-decomposition class for extracting features and reducing noise. The methods in the decomposition class assume that the target signal and the background noise can be separated into different frequency bands in the transform domain to realize the signal noise cancellation and extraction of wear particles. However, due to the wide frequency band of the wear particle induced voltage signals, part of which may be assigned to a noise frequency band, distortion and deformation of the wear particle induced voltage signals are bound to be caused when correlation coefficients of non-target signals are zeroed out. In particular, when a frequency band of target signals covers unwanted frequency components, the effectiveness and robustness of these algorithms are significantly reduced and even lead to a complete loss of the wear particle induced voltage signals. Non-decomposition algorithms, on the other hand, usually use an adaptive filter to improve a signal-to-noise ratio of the signals, but the distortion or the deformation of the wear particle induced voltage signals are still unavoidable in some cases, and residual random noise of filtered signals also will still affect the identification and the extraction of the wear particles.

Therefore, it is desired to provide a method for extracting a wear particle feature signal based on segmentation entropy, which better retains signal feature information of the wear particle while reducing noise.

### SUMMARY

One or more embodiments of the present disclosure provide a method for extracting a wear particle feature signal based on segmentation entropy. The method may include the following operations.

In S1, a raw signal to be processed may be obtained by performing real-time data acquisition of lubricating oil containing ferromagnetic wear particles using a lubricating oil wear particle monitoring system constructed based on an inductive particle detection sensor.

In S2, a preprocessed signal may be obtained by performing low-pass filtering and harmonic interference suppression on the raw signal to be processed.

In S3, the preprocessed signal may be divided into a plurality of time domain sequence segments with a sliding window with a fixed length and window shift, calculating segmentation entropy corresponding to each time domain sequence segment among the plurality of time domain sequence segments, and a segmentation entropy set may be normalized to obtain normalized segmentation entropy.

In S4, an empirical cumulative distribution of the normalized segmentation entropy may be obtained, an adaptive threshold may be obtained through curve fitting based on the empirical cumulative distribution, and a plurality of non-zero discrete time domain signal segments may be obtained by segmenting the preprocessed signal by the adaptive threshold.

In S5, a target signal feature recognition index of each non-zero discrete time domain signal segment among the plurality of non-zero discrete time domain signal segments may be calculated and an index threshold may be set, the non-zero discrete time domain signal segment may be excluded as residual noise interference when the target signal feature recognition index of the non-zero discrete time domain signal segment is less than the index threshold, and the non-zero discrete time domain signal segment may be retained when the target signal feature recognition index of the non-zero discrete time domain signal segment is not less than the index threshold to obtain a final extraction result of the wear particle feature signal.

In some embodiments, in S2, a low-pass filter may be configured to perform the low-pass filtering on the raw signal to be processed, a cutoff frequency f_{c} of the low-pass filter satisfies is larger than and equal to 2.5f_{d}, and f_{d} is a center frequency of a wear particle induced voltage signal.

In some embodiments, in S2, the harmonic interference suppression may be achieved by constructing harmonic components with opposite amplitudes but the same frequency and phase to be superimposed with the raw signal to be processed after the low-pass filtering, the frequency may be obtained by an iterative interpolation discrete Fourier transform algorithm, and the amplitudes and the phase may be obtained using a frequency domain compensation manner.

In some embodiments, S3 includes the following operations.

In S31, the preprocessed signal may be divided using the sliding window with a fixed window length N and a window shift Nₘ to obtain J-1 time domain sequence segments.

In S32, segmentation entropy of each time domain sequence segment may be calculated by an equation:

$${ζ}_{j} = N \times ln {S}_{N}$$

ζⱼ denotes segmentation entropy of j-th time domain sequence segment, and S_{N} denotes a sample variance of the j-th time domain sequence segment.

In S33, segmentation entropy corresponding to the J-1 time domain sequence segments may be normalized to obtain the normalized segmentation entropy by an equation:

$$\overline{ζ} = \frac{\hat{ζ}}{{‖ \hat{ζ} ‖}_{∞}}$$

\hat{ζ} denotes that a segmentation entropy set ζ={ζ₁, . . . , ζⱼ, . . . , ζ_{J-1}} is decentered, and ∥·∥∞ denotes an infinity norm. Elements in the normalized segmentation entropy ζ̅ take values within a range of [−1,1].

In S4, calculating the adaptive threshold and segmenting the preprocessed signal may include the following operations.

In S41, an interval [−1,1] may be delimited by a fixed step size μ, a random variable set X=[−1, −1+μ, . . . , 1] may be determined, sample point set R(X)=[R₋₁, R_{-1+μ}, . . . , R₁] in the normalized segmentation entropy which is smaller than a random variable xₘ (m=1, 2, . . . , 2/μ) may be counted one by one, and an empirical cumulative distribution R̅(X) of the normalized segmentation entropy may be determined using a normalization equation R̅(X)=R(X)/max (R(X)). max(·) denotes the maximum value in a set of extracted elements.

In S42, curve fitting may be performed using a Sigmoid function with a change trend similar to that of the empirical cumulative distribution R̅(X), an expression of the Sigmoid function may be:

$$S ( t ) = \frac{1}{1 + {e}^{- c t}}$$

c∈R+ denotes an adaptive tuning parameter, and an independent variable t takes the random variable set X as an input.

In S43, a curvature of the Sigmoid function may be calculated by an equation ρ(t)=|S″(t)|/[(1+S′(t))]^{3/2}, where S′(t) and S″(t) denote a first order derivative and a second order derivative of S(t), respectively, a random variable X may be input as an independent variable to obtain a curvature ρ(X), a mapping relationship with the random variable X as a horizontal coordinate and the curvature ρ(X) as a vertical coordinate may be established, and a horizontal coordinate random variable x_{ρmax} corresponding to a maximum of the curvature close to 1 may be designated as the adaptive threshold.

In S44, a preprocessed signal segment corresponding to a variable in the normalized segmentation entropy that is below the adaptive threshold x_{ρmax} may be set to zero and a preprocessed signal segment corresponding to a variable that is above the adaptive threshold x_{ρmax} may be retained to obtain H non-zero discrete time domain signal segments.

In some embodiments, in S5, for a non-zero discrete time domain signal segment Δ={Δ₀, Δ₁, . . . , Δ_{L-1}}, the target signal feature recognition index may include the following indices.

A. a time domain order index:

$$δ = \{ \begin{matrix}1 , & Ω > 0 \\ 0 , & Ω < 0\end{matrix}$$

Ω=Lₘ−Lₙ. Lₘ and Lₙ denote a position of a horizontal coordinate corresponding to a maximum value of a signal amplitude and a position of a horizontal coordinate corresponding to a minimum value of the signal amplitude in the non-zero discrete time domain signal segment, respectively. δ=1 denotes that the non-zero discrete time domain signal segment conforms to a morphology feature of the wear particle induced voltage signal. δ=0 denotes that the non-zero discrete time domain signal segment may be directly excluded as a non-wear particle induced voltage signal.

### B. a Marginal Characterization Index:

$$ς = Q ( \frac{{L}_{n}}{Ω} ) \times Q ( \frac{L - {L}_{m}}{Ω} )$$

Q(·) denotes a function on ε defined by:

$$Q ( ε ) = \{ \begin{matrix}1 , & ε \geq 1 \\ ε , & ε < 1\end{matrix}$$

ζ≈1 denotes that there is a higher probability that the non-zero discrete time domain signal segment has a marginal feature of the wear particle induced voltage signal. ζ<<1 denotes that the non-zero discrete time domain signal segment may be classified as the non-wear particle induced voltage signal.

### C. An Unbiasedness Index:

$$γ = 1 - \frac{\vert {\sum }_{i - {L}_{n}}^{{L}_{m}} {Δ}_{i} \vert}{{\sum }_{i = {L}_{n}}^{{L}_{m}} \vert {Δ}_{i} \vert}$$

Δᵢ denotes i-th element in the non-zero discrete time domain signal segment Δ. γ takes a value within a range of [0,1]. A lower value of γ indicates a higher bias of the non-zero discrete time domain signal segment and a higher probability of the non-zero discrete time domain signal segment being a non-wear particle signal, and the non-zero discrete time domain signal segment with the lower value of γ may be excluded, and conversely, non-zero discrete time domain signal segment may be retained.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which like reference numerals represent similar structures, and wherein:

FIG. 1 is a flowchart illustrating an algorithm according to some embodiments of the present disclosure;

FIG. 2 is a schematic diagram illustrating a platform of a lubricating oil wear particle monitoring system according to some embodiments of the present disclosure;

FIG. 3 is a schematic diagram illustrating a raw signal to be processed extracted from a lubricating oil wear particle monitoring system according to some embodiments of the present disclosure;

FIG. 4 is a schematic diagram illustrating a preprocessed signal for low-pass filtering and harmonic interference suppression according to some embodiments of the present disclosure;

FIG. 5 is a schematic diagram illustrating a result of normalized segmentation entropy of a preprocessed signal according to some embodiments of the present disclosure;

FIG. 6 is a schematic diagram illustrating a result of an empirical cumulative distribution R(X), a Sigmoid function fitted waveform S(X), and a corresponding curvature ρ(X) in a process of determining an adaptive threshold according to some embodiments of the present disclosure;

FIG. 7 is a schematic diagram illustrating a result of segmenting a preprocessed signal by an adaptive threshold according to some embodiments of the present disclosure;

FIG. 8 is a schematic diagram illustrating a result of depth recognition of a target recognition feature index according to some embodiments of the present disclosure;

FIG. 9 is a partially enlarged view of wear particle induced voltage signals retained by an algorithm according to some embodiments of the present disclosure; and

FIG. 10 is a schematic diagram illustrating an exemplary threshold determination model according to some embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “system”, “device”, “unit” and/or “module” as used herein is a manner used to distinguish different components, elements, parts, sections, or assemblies at different levels. However, if other words serve the same purpose, the words may be replaced by other expressions.

As shown in the present disclosure and claims, the words “one”, “a”, “a kind” and/or “the” are not especially singular but may include the plural unless the context expressly suggests otherwise. In general, the terms “comprise”, “comprises”, “comprising”, “include”, “includes”, and/or “including”, merely prompt to include operations and elements that have been clearly identified, and these operations and elements do not constitute an exclusive listing. The methods or devices may also include other operations or elements.

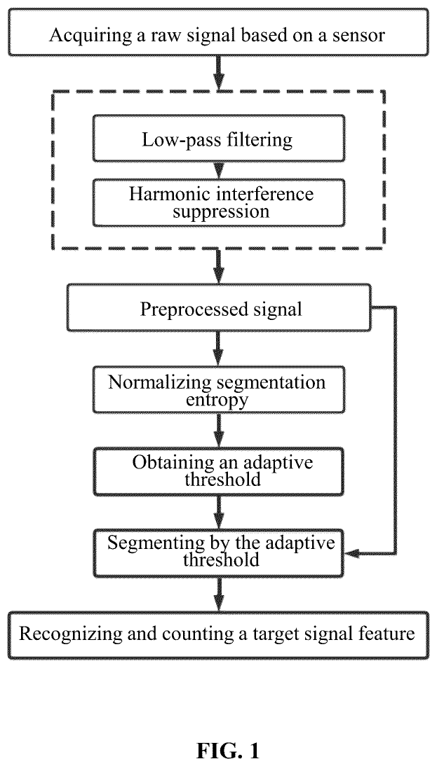

FIG. 1 is a flowchart illustrating an algorithm according to some embodiments of the present disclosure.

Some embodiments of the present disclosure provide a method for extracting a wear particle feature signal based on segmentation entropy. As shown in FIG. 1, the method includes the following operations.

In S1, a raw signal to be processed is obtained by performing real-time data acquisition of lubricating oil containing ferromagnetic wear particles using a lubricating oil wear particle monitoring system constructed based on an inductive particle detection sensor.

The inductive particle detection sensor refers to a sensor configured to detect wear particles in the fluid (e.g., the lubricating oil). For example, the inductive particle detection sensor may be a single-excitation inductive particle sensor. The inductive particle detection sensor is mainly based on a principle of electromagnetic induction. When metal wear particles in the fluid (e.g., the lubricating oil) pass through the sensor, a distribution of magnetic field around a coil inside the sensor is changed. The change of the magnetic field in the coil leads to a change of an induced electromotive force, and the change of the induced electromotive force is detected by the sensor and converted into an electric signal. The electric signal is amplified and processed, and the sensor ultimately outputs a signal (e.g., the raw signal to be processed) related to a count and a size of the wear particles.

The lubricating oil wear particle monitoring system refers to a system for monitoring the condition of wear particles in the lubricating oil. In some embodiments, the lubricating oil wear particle monitoring system may include one or more inductive particle detection sensors. In some embodiments, the lubricating oil wear particle monitoring system may also include a processor for processing relevant data (e.g., data collected by the inductive particle detection sensor).

The raw signal to be processed refers to an unprocessed initial signal containing raw information about the wear particles in the lubricating oil.

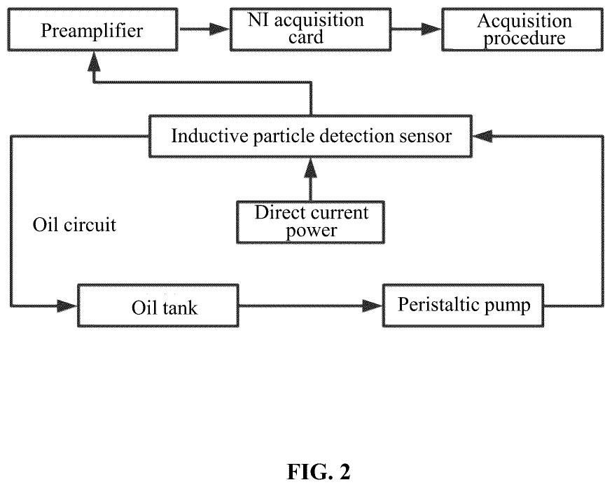

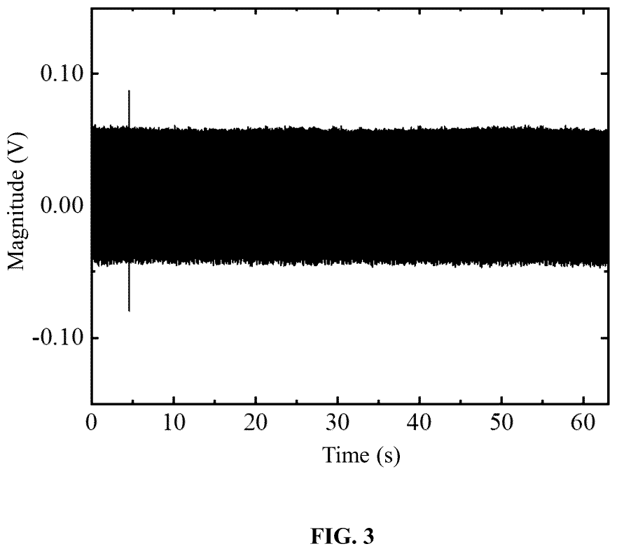

FIG. 2 is a schematic diagram illustrating a platform of a lubricating oil wear particle monitoring system according to some embodiments of the present disclosure. FIG. 3 is a schematic diagram illustrating a raw signal to be processed extracted from the lubricating oil wear particle monitoring system according to some embodiments of the present disclosure.

Merely by way of example, a process by which the lubricating oil wear particle monitoring system acquires raw signals is shown as below.

A schematic diagram of the platform of the lubricating oil wear particle monitoring system is constructed as shown in FIG. 2. A model of an acquisition card is NI-9219, a sampling frequency fₛ is set to 5000 Hz, and a count of sample points N is 315000. The inductive particle detection sensor adopts direct current excitation, a driving current I is 0.5 A, an amplifier multiplier of a preamplifier is 2000, and a flow rate of a peristaltic pump is set to 510 ml/min. The wear particles pass through the inductive particle detection sensor with a size (an equivalent diameter of a sphere) of 220 mm, 110 mm, 50 mm, 150 mm, 65 mm, 130 mm, 80 mm, and 180 mm in sequence, and the inductive particle detection sensor generates 8 inductive voltage signals similar to a single-cycle sinusoidal waveform. The raw signal to be processed is as shown in FIG. 3, which is seen to contain a large amount of interfering noise, and it is not possible to quickly and accurately extract morphology features of tiny wear particle induced voltage signals.

The wear particle induced voltage signal refers to a voltage signal generated when the inductive particle detection sensor detects the wear particles in the lubricating oil. The morphology feature of the wear particle induced voltage signal refers to the shape and the feature of the wear particle induced voltage signals, which is used to distinguish different types of the wear particles. For example, the morphology features may include an amplitude, a frequency, a phase, a pulse width, and a pulse shape.

In S2, a preprocessed signal is obtained by performing low-pass filtering and harmonic interference suppression on the raw signal to be processed.

The preprocessed signal refers to a signal after removing high-frequency background noise and low-frequency harmonic interference and retaining effective features of the wear particle induced voltage signal. In some embodiments, the preprocessed signal may be obtained by preprocessing (e.g., the low-pass filtering and the harmonic interference suppression) the raw signal to be processed.

Because the raw signal to be processed acquired by the lubricating oil wear particle monitoring system in real-time not only contains the wear particle induced voltage signals, but also harmonic interference caused by structural vibrations and random noises caused by electromagnetic waves, which overwhelm the wear particle induced voltage signals needed and therefore need to be removed as much as possible.

In some embodiments, in S2, a low-pass filter is configured to perform the low-pass filtering on the raw signal to be processed, and a cutoff frequency f_{c} of the low-pass filter satisfies is larger than and equal to 2.5 f_{d}, and f_{d} is a center frequency of the wear particle induced voltage signal. Understandably, by limiting the cutoff frequency of the low-pass filter, it is helpful to ensure that the low-pass filter can effectively filter out high-frequency noise while retaining the main frequency components of the wear particle induced voltage signals.

In some embodiments, in S2, the harmonic interference suppression is achieved by constructing harmonic components with opposite amplitudes but the same frequency and phase to be superimposed with the raw signal to be processed after the low-pass filtering. The frequency is obtained by an iterative interpolation discrete Fourier transform algorithm, and the amplitudes and the phase are obtained using a frequency domain compensation manner.

Merely by way of example, the preprocessing process may include the following operations.

For an acquired raw signal to be processed, the low-pass filter is first used to filter the high-frequency background noise with a cutoff frequency f_{c} of 130 Hz. Then an amplitude, a frequency, and a phase of each low-frequency harmonic interference are computed by the iterative interpolation discrete Fourier transform algorithm (e.g., a matrix multiplication and a fast Fourier transform (FFT), etc.) and the frequency domain compensation manner (e.g., a frequency-domain equalization algorithm, an adaptive equalization algorithm, and a frequency-domain correction and compensation algorithm), respectively. Finally, harmonic estimation components with opposite amplitudes but the same frequency and phase are superimposed with the raw signal to be processed after the low-pass filtering, to achieve the purpose of low-frequency harmonic interference suppression.

The harmonic estimation components refer to signals used to counteract the harmonic interference. In some embodiments, a superposition of the harmonic estimation components with the raw signal to be processed may be a superposition of an algebraic sum superposition, a weighted superposition, etc. The weight of the weighted superposition may be determined based on amplitude features or frequency features of the signal. For example, the weight may be set based on the degree of influence of the harmonic component on oil wear particle induced voltage signals, and a greater weight may be assigned to the harmonic component with a greater influence.

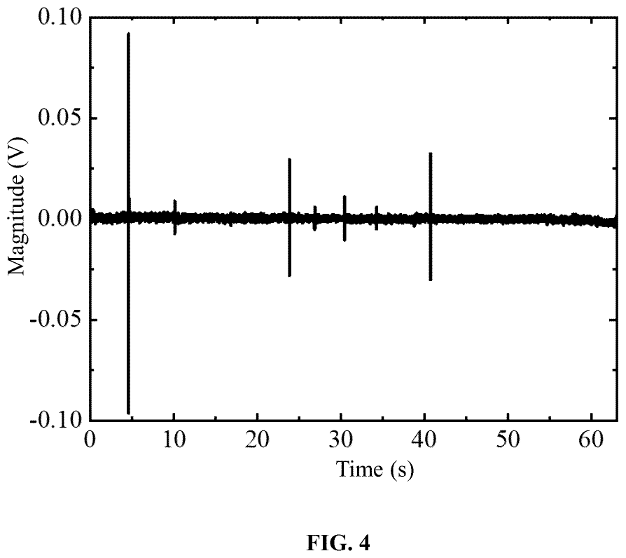

FIG. 4 is a schematic diagram illustrating a preprocessed signal for low-pass filtering and harmonic interference suppression according to some embodiments of the present disclosure.

A preprocessing result of a signal is shown in FIG. 4. It is observed that the harmonic interference and the random noises of the raw signal to be processed are effectively suppressed, and a signal-to-noise ratio of the signal is obviously improved. However, a large amount of low-frequency random noise is still contained, which seriously impedes the wear particle induced voltage signals.

In S3, the preprocessed signal is divided into a plurality of time domain sequence segments with a sliding window with a fixed length and window shift, segmentation entropy corresponding to each time domain sequence segment among the plurality of time domain sequence segments is calculated, and a segmentation entropy set is normalized to obtain normalized segmentation entropy.

The time domain sequence segments refer to a plurality of localized data segments obtained by dividing the preprocessed signal on a time axis according to the sliding window with the fixed length and window shift. The segmentation entropy set refers to a set of values of the segmentation entropy corresponding to each time domain sequence segment. The normalized segmentation entropy refers to a result obtained by normalizing each value of the segmentation entropy in the segmentation entropy set. In some embodiments, the fixed length may be determined by the processor based on a feature frequency of the signal or an expected duration of an oil wear particle event. For example, by analyzing historical data or performing an experimental calibration, a window length that covers features of a complete wear particle event is selected.

In some embodiments, a process of determining the normalized segmentation entropy includes the following operations.

In S31, the preprocessed signal is divided using the sliding window with a fixed window length N and window shift Nₘ to obtain J-1 time domain sequence segments.

In S32, the segmentation entropy of each time domain sequence segment is calculated by an equation:

$${ζ}_{j} = N \times ln {S}_{N} . \qquad ( 1 )$$

In the equation (1), ζⱼ denotes segmentation entropy of j-th time domain sequence segment, and S_{N} denotes a sample variance of the j-th time domain sequence segment.

In S33, segmentation entropy corresponding to the J-1 time domain sequence segments is normalized to obtain the normalized segmentation entropy by an equation:

$$\overline{ζ} = \frac{\hat{ζ}}{{‖ \hat{ζ} ‖}_{∞}} . \qquad ( 2 )$$

In the equation (2), \hat{ζ} denotes that a segmentation entropy set ζ={ζ₁, . . . , ζⱼ, . . . , ζ_{J-1}} is decentered, ∥·∥∞ denotes an infinity norm of the segmentation entropy set, and elements in the normalized segmentation entropy ζ̅ take values within a range of [−1,1].

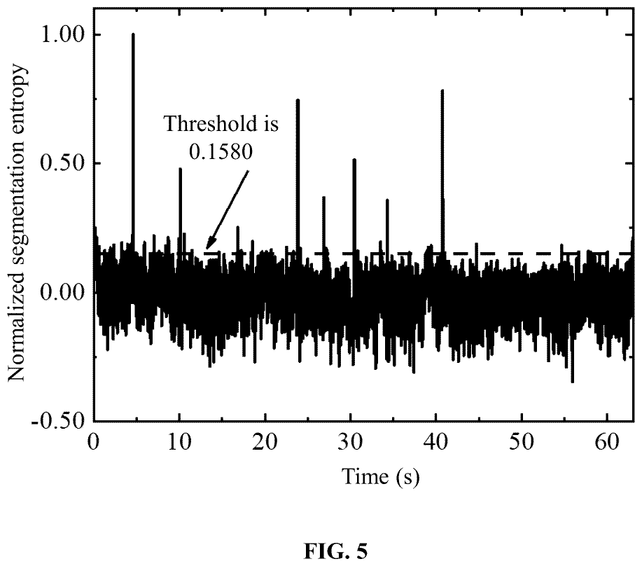

FIG. 5 is a schematic diagram illustrating a result of normalized segmentation entropy of a preprocessed signal according to some embodiments of the present disclosure.

Merely by way of example, in the above operations, N=150, Nₘ=1, and a result of the normalized segmentation entropy calculated based on the above operations is shown in FIG. 5. The result shows that the low-frequency random noise superimposed on the wear particle induced voltage and an electrical impulse is significantly higher than that of a portion that contains only the low-frequency random noise. Due to the difference, the signal may be segmented according to a reasonable threshold, thereby realizing the localization of the wear particle induced voltage signal.

In S4, an empirical cumulative distribution of the normalized segmentation entropy is obtained, an adaptive threshold through curve fitting is obtained based on the empirical cumulative distribution, and a plurality of non-zero discrete time domain signal segments is obtained by segmenting the preprocessed signal by the adaptive threshold.

The adaptive threshold refers to a threshold that is dynamically determined based on a plurality of features and used for signal segmentation during signal processing.

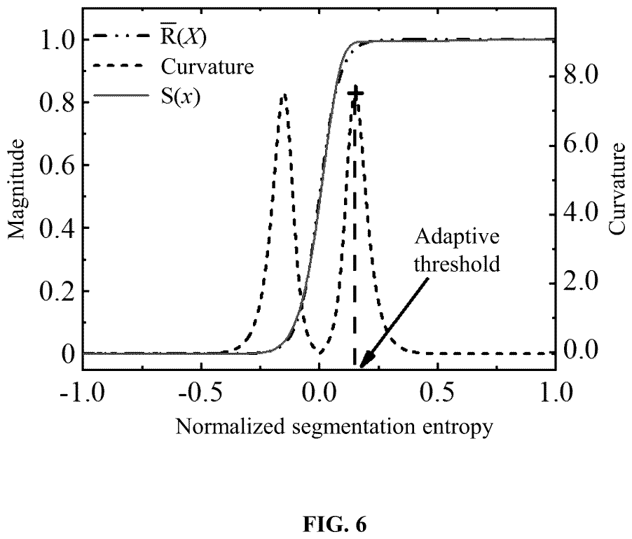

FIG. 6 is a schematic diagram illustrating a result of an empirical cumulative distribution R̅(X), a Sigmoid function fitted waveform S(X), and a corresponding curvature ρ(X) in a process of determining an adaptive threshold according to some embodiments of the present disclosure.

In some embodiments, calculating the adaptive threshold and segmenting the preprocessed signal includes the following operations.

In S41, an interval [−1,1] is delimited by a fixed step size μ, a random variable set X=[−1, −1+μ . . . , 1] is determined, sample point set R(X)=[R₋₁, R_{-1+μ}, . . . , R₁] in the normalized segmentation entropy which is smaller than a random variable xₘ (m=1, 2, . . . , 2/m) is counted one by one, and an empirical cumulative distribution R̅(X) of the normalized segmentation entropy is determined using a normalization equation. For example, the normalization equation is:

$$\overline{R} ( X ) = R ( X ) / max ( R ( X ) ) . \qquad ( 3 )$$

In the equation (3), max (R(X)) denotes the maximum value in the sample point set R(X).

Merely by way of example, μ=1/1000 and m=2000, and a curve of an empirical cumulative distribution R̅(X) is obtained as shown by a double dotted line in FIG. 6.

In S42, curve fitting is performed using a Sigmoid function with a change trend similar to that of the empirical cumulative distribution R̅(X), an expression of the Sigmoid function is:

$$S ( t ) = \frac{1}{1 + {e}^{- ct}} . \qquad ( 4 )$$

In the equation (4), c∈R+ is an adaptive tuning parameter that may be approximated by taking a first order derivative of S(t) and substituting t=0 to estimate the parameter c=4k₀. k₀ denotes a slope of discrete points of the empirical cumulative distribution R̅(X) at a random variable x=0, which is estimated from elements of the empirical cumulative distribution R̅(X), and in this embodiment k₀=4.13. When an independent variable t takes a random variable set X as an input, a fitted curve S(X) is determined as shown by a solid line in FIG. 6.

In S43, a curvature of the Sigmoid function is calculated by an equation ρ(t)=|S″(t)|/[(1+S′(t))]^{3/2}. S′(t) and S″(t) denote a first order derivative and a second order derivative of S(t), respectively. The curvature ρ(X) may be obtained by inputting a random variable X as an independent variable, as shown by a dashed line in FIG. 6. A mapping relationship with the random variable X as a horizontal coordinate and the curvature ρ(X) as a vertical coordinate is established, and a horizontal coordinate random variable x_{ρmax} corresponding to the maximum value of the curvature close to 1 is designated as the adaptive threshold. For example, the horizontal coordinate random variable x_{ρmax} of 0.1580 is designated as the adaptive threshold.

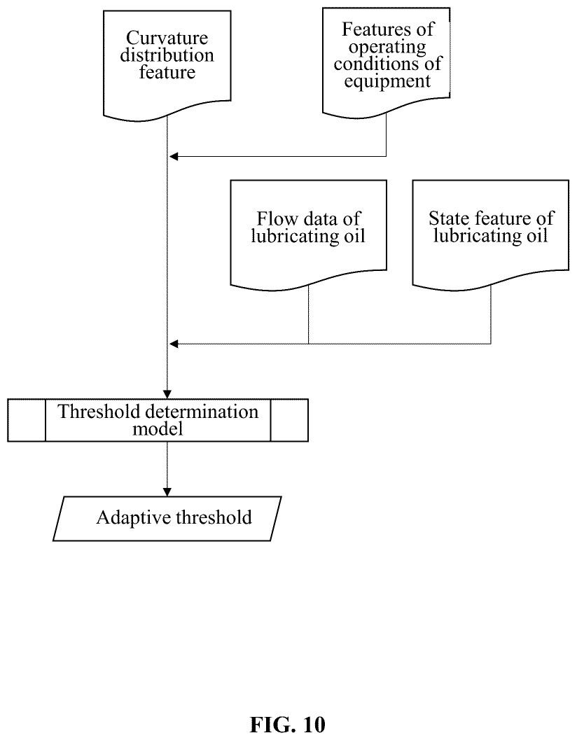

More descriptions regarding determining the adaptive threshold may be found in FIG. 10 and related descriptions thereof.

In some embodiments of the present disclosure, a method for determining the adaptive threshold based on Sigmoid curve fitting is capable of adaptively adjusting the threshold based on empirical cumulative distributions of different normalized segmentation entropy, so that the adaptive threshold is consistent with statistical distribution features of data processed, thereby achieving effects of the best noise reduction and the best wear particle retention, and exhibiting strong robustness.

In S44, a preprocessed signal segment corresponding to a variable in the normalized segmentation entropy that is below the adaptive threshold x_{ρmax} is set to zero and a preprocessed signal segment corresponding to a variable that is above the adaptive threshold x_{ρmax} is retained to obtain H non-zero discrete time domain signal segments.

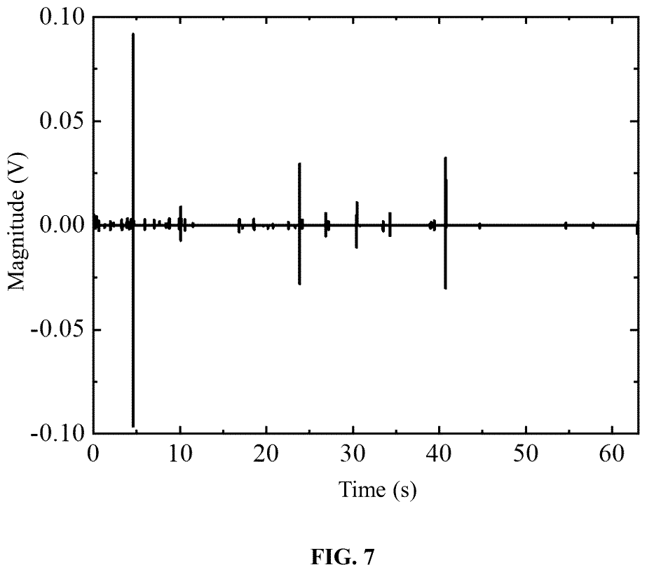

FIG. 7 is a schematic diagram illustrating a result of segmenting a preprocessed signal by an adaptive threshold according to some embodiments of the present disclosure.

In some embodiments, the normalized segmentation entropy and the preprocessed signal are in a one-to-one correspondence. A corresponding part of the normalized segmentation entropy lower than the adaptive threshold is not satisfied in the preprocessed signal, so that the preprocessed signal is directly zeroed. The result of 54 non-zero discrete time domain signal segments is shown in FIG. 7. It is seen that a segmentation result of the preprocessed signal can achieve the purpose of preservation of a wear particle feature and noise reduction.

In S5, a target signal feature recognition index of each non-zero discrete time domain signal segment among the plurality of non-zero discrete time domain signal segments is calculated and an index threshold is set, the non-zero discrete time domain signal segment is excluded as residual noise interference when the target signal feature recognition index of the non-zero discrete time domain signal segment is less than the index threshold, and the non-zero discrete time domain signal segment is retained when the target signal feature recognition index of the non-zero discrete time domain signal segment is not less than the index threshold to obtain a final extraction result of the wear particle feature signal.

The target signal feature recognition index refers to a characteristic parameter used to determine whether the non-zero discrete time domain signal segment is a valid wear particle signal. The index threshold refers to a preset value used to determine whether the target signal feature recognition index satisfies a reservation condition. In some embodiments, the index threshold may be determined based on historical data and experience.

In some embodiments, after threshold segmentation, the signal still has interfering noise, and the interfering noise is further ruled out using the target signal feature recognition index. In S5, for a non-zero discrete time domain signal segment Δ={Δ₀, Δ₁, . . . , Δ_{L-1}}, the target signal feature recognition index includes the following indices.

### A. a Time Domain Order Index:

$$δ = \{ \begin{matrix}1 , \\ 0 ,\end{matrix} \begin{matrix}Ω > 0 \\ Ω < 0\end{matrix} . \qquad ( 5 )$$

In the equation (5), Ω=Lₘ-Lₙ. Lₘ and Lₙ denote a position of a horizontal coordinate corresponding to a maximum value of a signal amplitude and a position of a horizontal coordinate corresponding to a minimum value of the signal amplitude in the non-zero discrete time domain signal segment, respectively. δ=1 denotes that the non-zero discrete time domain signal segment conforms to a morphology feature of the wear particle induced voltage signal. δ=0 denotes that the non-zero discrete time domain signal segment is directly excluded as a non-wear particle induced voltage signal.
B. A marginal Characterization Index:

$$ϛ = Q ( \frac{{L}_{n}}{Ω} ) \times Q ( \frac{L - {L}_{m}}{Ω} ) . \qquad ( 6 )$$

In the equation (6), Q(·) denotes a function on & defined by:

$$Q ( ε ) = \{ \begin{matrix}1 , & ε \geq 1 \\ ε , & ε < 1\end{matrix} . \qquad ( 7 )$$

In the equation (7), ζ≈1 denotes that there is a higher probability that the non-zero discrete time domain signal segment has a marginal feature of the wear particle induced voltage signal. ζ<<1 denotes that the non-zero discrete time domain signal segment is classified as the non-wear particle induced voltage signal.

### C. An Unbiasedness Index:

$$γ = 1 - \frac{\vert {\sum }_{i = {L}_{n}}^{{L}_{m}} {Δ}_{i} \vert}{{\sum }_{i = {L}_{n}}^{{L}_{m}} \vert {Δ}_{i} \vert} . \qquad ( 8 )$$

In the equation (8), Δᵢ denotes the i-th element in the non-zero discrete time domain signal segment Δ, and γ takes a value within a range of [0,1]. A lower value of γ indicates a higher bias of the non-zero discrete time domain signal segment and a higher probability of the non-zero discrete time domain signal segment being a non-wear particle signal, and the non-zero discrete time domain signal segment with the lower value of γ may be excluded, and conversely, non-zero discrete time domain signal segment may be retained.

In some embodiments, the non-zero discrete time domain signal segment is screened by the time domain order index δ. The interfering noise exits leads to a result of δ=0, and a corresponding non-zero time domain signal segment may be directly set to zero. 27 non-zero time domain signal segments retained by δ=1 need to be further identified by the marginal characterization index ζ and the unbiasedness index γ, and corresponding calculation results are shown in tables 1 and 2. It can be seen by concatenated multiplication values that calculation results of the wear particle induced voltage signal are significantly higher than that of noise segment. Therefore, an index threshold of 0.7 may be further set to exclude spurious wear particle signals, i.e., a non-zero discrete time domain signal segment that is less than the index threshold is set to zero. Conversely, a non-zero discrete time domain signal segment that is more than the index threshold is retained.

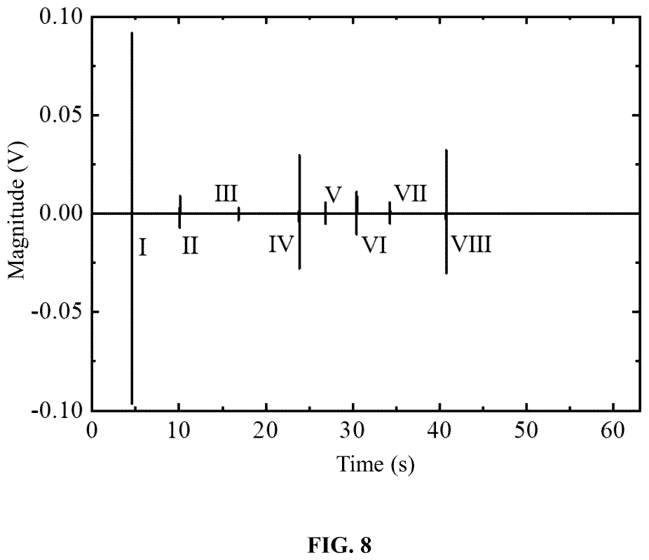

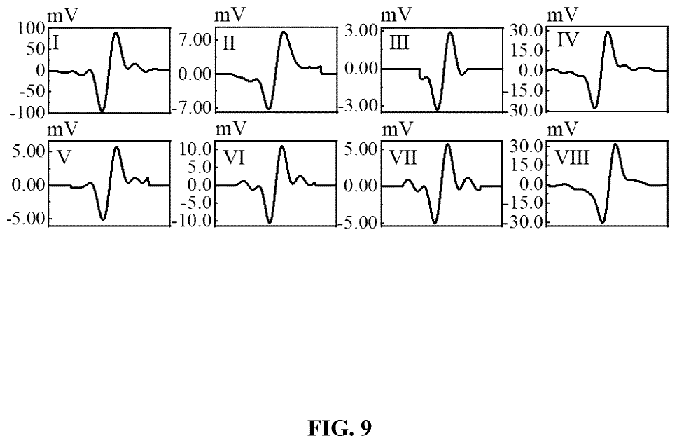

FIG. 8 is a schematic diagram illustrating a result of depth recognition of a target recognition feature index according to some embodiments of the present disclosure. FIG. 9 is a partially enlarged view of wear particle induced voltage signals retained by an algorithm according to some embodiments of the present disclosure.

A final result of the raw signal to be processed by the above operations is shown in FIG. 8, it is seen that a signal of the interfering noise has been better suppressed. From the partially enlarged view of the wear particle induced voltage signals in FIG. 9, it is also be seen that the morphology features of the wear particle signal are well preserved, which may be used for counting and calibrating the wear particle signal. The purpose of the counting is to count and sort the wear particles, making it easy to analyze the wear particles individually subsequently. The counting is a preliminary operation of calibration of the wear particles.

TABLE 1
The wear particle signal feature recognition index

| Order | I | II | III | IV | V | VI | VII | VIII |
|---|---|---|---|---|---|---|---|---|
| ζ | 1.00 | 1.00 | 1.00 | 1.00 | 1.00 | 1.00 | 1.00 | 1.00 |
| g | 0.99 | 0.84 | 0.90 | 0.98 | 0.95 | 0.96 | 0.87 | 0.99 |
| ζ × γ | 0.99 | 0.84 | 0.90 | 0.98 | 0.95 | 0.96 | 0.87 | 0.99 |

TABLE 2
Noise interference feature recognition index

| Order | 01 | 02 | 03 | 04 | 05 | 06 | 07 | 08 | 09 |
|---|---|---|---|---|---|---|---|---|---|
| ζ | 0.26 | 0.11 | 1.00 | 0.02 | 0.04 | 0.20 | 0.03 | 0.59 | 0.02 |
| g | 0.17 | 0.08 | 0.32 | 0.17 | 0.00 | 0.00 | 0.13 | 0.39 | 0.06 |
| ζ × γ | 0.04 | 0.01 | 0.32 | 0.00 | 0.00 | 0.00 | 0.00 | 0.23 | 0.00 |
| Order | 10 | 11 | 12 | 13 | 14 | 15 | 16 | 17 | 18 |
| ζ | 0.02 | 0.11 | 0.07 | 0.58 | 0.11 | 0.87 | 0.20 | 0.43 | 0.06 |
| g | 0.44 | 0.55 | 0.00 | 0.00 | 0.93 | 0.30 | 0.21 | 0.00 | 0.37 |
| ζ × γ | 0.01 | 0.06 | 0.00 | 0.00 | 0.11 | 0.26 | 0.04 | 0.00 | 0.02 |
| Order | 19 | 20 | 21 | 22 | 23 | 24 | 25 | 26 | 27 |
| ζ | 0.02 | 0.01 | 0.08 | 0.17 | 0.83 | 0.43 | 0.11 | 0.25 | 0.04 |
| g | 0.38 | 0.47 | 0.00 | 0.72 | 0.00 | 0.74 | 0.01 | 0.01 | 0.21 |
| ζ × γ | 0.01 | 0.01 | 0.00 | 0.12 | 0.00 | 0.32 | 0.00 | 0.00 | 0.01 |

The algorithm provided in some embodiments of the present disclosure can realize background noise suppression while protecting morphology features of tiny wear particle signals from being destroyed. Traditional methods of noise reduction may result in distortion of the target signal feature or residual excessive interfering noise due to decomposition problems or lack of post-processing, which is not conducive to statistics and calibration of the wear particles.

The algorithm provided in some embodiments of the present disclosure detects the wear particle signal by the segmentation entropy based on the variability of components of a signal, which avoids distortion of the wear particle signal and amplitude degradation due to an error of frequency decomposition. In addition, through the threshold segmentation and further depth recognition of the target signal, not only the morphology features of the wear particle induced voltage signals are retained better, but also most of the residual interfering noise can be excluded, thereby realizing an accurate recognition and extraction of the wear particle signal.

The algorithm provided in some embodiments of the present disclosure has a good migration effect. When an output signal is smooth, an object targeted by a determination and segmentation method based on signal preprocessing, normalized segmentation entropy detection, and adaptive threshold is not limited to a signal generated by the inductive particle detection sensor. Rather, the method may be applied to detection of other weak target signals similar to mutant pulses.

In some embodiments, the method for extracting the wear particle feature signal based on the segmentation entropy further includes determining, at a preset interval, a plurality of target wear parts based on a plurality of extraction results of the wear particle feature signal, and generating a plurality of part regulation instructions to control the plurality of target wear parts to operate based on the plurality of part regulation instructions.

The preset interval refers to a pre-determined time interval used to periodically determine the target wear parts and generate the part regulation instruction. In some embodiments, the preset interval may be determined manually based on equipment operating conditions, maintenance needs, or experience.

The wear particle feature refers to a feature of the lubricating oil that reflects the wear condition of mechanical equipment. For example, the wear particle feature may include a size, a shape, and a count of the wear particles. The plurality of extraction results of the wear particle feature signal refer to a set of the wear particle feature signal collected by the plurality of inductive particle detection sensors and obtained by feature extraction during a previous preset interval. The extraction results of the wear particle feature signal may include a count and a size of the wear particles in the lubricating oil. In some embodiments, the extraction results of the wear particle feature signal may reflect the wear condition of equipment.

More descriptions regarding obtaining the extraction results of the wear particle feature signal may be found in FIGS. 1-9 and related descriptions thereof.

The target wear part refers to a part of which a wear degree beyond a wear degree threshold in the mechanical equipment. The wear degree threshold refers to a preset standard value for determining the wear degree of a part of the mechanical equipment. The wear degree threshold may be set manually based on experience.

The processor may determine the plurality of target wear parts in a plurality of ways based on the plurality of extraction results of the wear particle feature signal.

In some embodiments, the processor may perform a cluster analysis on each wear particle feature signal in the extraction results of the wear particle feature signal to match to obtain a wear part corresponding to each wear particle feature signal. Exemplarily, the processor may construct a target vector based on each wear particle feature signal of the plurality of extraction results of the wear particle feature signal, construct a clustering vector based on historical wear particle feature signals in historical data of the mechanical equipment, label the clustering vector as wear parts actually corresponding to the historical wear particle feature signals corresponding to the clustering vector, obtain a plurality of clustering clusters by clustering based on a clustering index, filter out a clustering cluster (denoted as a target cluster) which contain the target vector, and designate a label (i.e., the wear part with the highest percentage) corresponding to all the clustering vectors in the target clusters as the wear part corresponding to the target vector. The historical wear particle feature signals may be obtained based on historical data, and labels of the clustering vectors may be obtained from historical maintenance records.

In some embodiments, the processor may count a count of wear particles corresponding to each wear part based on the plurality of wear parts corresponding to the plurality of wear particle feature signals and determine the wear part with a count of wear particles that exceeds a preset quantity threshold as a target wear part. The preset quantity threshold may be set based on experience.

The part regulation instruction refers to a related instruction used to control the operation of the target wear parts. In some embodiments, the part regulation instruction includes an operating power and/or a rotational speed of the part.

The operating power of the part refers to the power to drive the target wear parts. The rotational speed of the part refers to the count of revolutions per unit time that the target wear parts rotate.

The processor may generate the part regulation instructions in a plurality of ways. In some embodiments, the processor may generate the plurality of part regulation instructions based on the wear degree of the target wear parts and a related regulation strategy. For example, for a certain target wear part, when the extraction results of the wear particle feature signal show that the wear degree of the target wear part exceeds the wear degree threshold, a part regulation instruction that reduces the operating power and the rotational speed of the part by a preset reduction magnitude may be generated. Merely by way of example, assume that a part of equipment normally operates with an operating power of P1 and a rotational speed of NI, and a wear rate of the part may be reduced by adjusting the operating power to P2 (P2=P1 (1−x1)) and the rotational speed to N2 (N2=N1 (1−x2)) according to a preset regulation strategy. x1 and x2 are the preset reduction magnitudes of the operating power and the rotational speed of the part, respectively. x1 and x2 may be preset manually based on experience.

In some embodiments, the processor may determine, by querying a preset table, the operating power and/or the rotational speed of each target wear part based on the plurality of target wear parts and automatically generate the part regulation instruction including the operating power and/or the rotational speed based on the operating power and/or the rotational speed. The preset table includes a correspondence between the target wear parts, the count of wear particles corresponding to the target wear parts, the operating power, and the rotational speed of the part. The preset table may be set by a technician according to need.

In some embodiments, in response to receiving the part regulation instruction, the target wear parts may adjust operating parameters of the target wear parts via a control system to match the operating power and/or the rotational speed of the part set in the part regulation instruction. For example, when a bearing component receives an instruction to reduce the rotational speed, an internal control system may adjust a frequency or torque of a drive motor to reduce the rotational speed, thereby reducing wear and the risk of failure. The control system may be a programmable logic controller (PLC), a microcontroller unit (MCU), a distributed control system (DCS), or a customized electronic control unit.

In some embodiments of the present disclosure, by determining the target wear parts based on the extraction results of the wear particle feature signal and generating a corresponding part regulation instruction, the wear of the part may be effectively slowed down. When the extraction results of the wear particle feature signal indicate that a certain part have a high wear degree, appropriately lowering the operating power and/or rotational speed of the part may reduce friction and impact force between the parts, which helps to reduce an operating pressure of the parts, which in turn reduces a probability of sudden failure of the equipment when the equipment is in a high wear state, and improves stability and reliability of operation of the equipment.

In some embodiments, the method for extracting the wear particle feature signal based on the segmentation entropy further includes determining, at a preset interval, a wear particle feature and a wear particle discrepancy feature in the lubricating oil based on the extraction results of the wear particle feature signal, and generating a detection and regulation instruction based on the wear particle feature and the wear particle discrepancy feature to control the inductive particle detection sensor to perform data acquisition based on the detection and regulation instruction.

The wear particle discrepancy feature refers to a size distribution of the plurality of the wear particles in the lubricating oil. For example, sizes of wear particles A, B, and Care 1 μm, 1 μm, and 2 μm, respectively, wear particle discrepancy features of the wear particles A, B, and C may be expressed as ((size: 1 μm, a count of the wear particle: 2) and (size: 2 μm, a count of the wear particle: 1)).

In some embodiments, the processor may obtain information such as the size of the wear particles and the count of the wear particles from the extraction results of the wear particle feature signal, so as to determine the wear particle feature, and determine the wear particle discrepancy feature by counting a count distribution of the wear particles of different sizes.

The detection and regulation instruction refers to an instruction related to controlling the inductive particle detection sensor for data acquisition. In some embodiments, the detection and regulation instruction include a detection frequency and a sampling amount.

The detection frequency refers to a count of times the lubricating oil is tested for wear particles per unit time. The sampling amount refers to a count of data points acquired during each detection.

In some embodiments, the processor may determine and generate the detection and regulation instruction based on the wear particle feature and the wear particle discrepancy feature in the oil via a first vector database.

The first vector database stores a plurality of feature vectors constructed from different historical wear particle features, historical wear particle discrepancy features, and labels corresponding to the feature vectors. The labels corresponding to the feature vectors include a preferred detection frequency and a preferred sampling amount. In some embodiments, the processor may designate a historical detection frequency and a historical sampling amount that minimizes a difference in the count of wear particles in a plurality of historical detections corresponding to a certain feature vector as a label corresponding to that feature vector. The difference in the count of wear particles refers to an extreme difference in the count of wear particles extracted from a plurality of signal acquisitions in the historical detection.

In some embodiments, the processor may construct a to-be-matched vector based on the wear particle feature and the wear particle discrepancy feature in the oil, and based on the to-be-matched vector, determine the feature vector that has the highest similarity to the to-be-matched vector by performing a vector search in the first vector database, and designate a label corresponding the feature vector as a detection frequency and a sampling amount corresponding to the to-be-matched vector, thereby generating the detection and regulation instruction. The way for determining the similarity includes, but is not limited to, a Euclidean distance, a cosine similarity, etc.

In some embodiments, in response to receiving the detection and regulation instruction, the inductive particle detection sensor may perform the data acquisition according to the detection frequency and the sampling amount in the detection and regulation instruction.

In some embodiments of the present disclosure, by determining the wear particle feature and the wear particle discrepancy feature in the lubricating oil based on the extraction results of the wear particle feature signal and generating the detection and regulation instruction that includes the detection frequency and the sampling amount, it is possible to realize a precise regulation of the data acquisition process of the inductive particle detection sensor. Feature-based dynamic adjustment mechanism enables the detection frequency and the sampling amount to automatically adapt to changes of the wear particle features of the lubricating oil, thereby improving accuracy and efficiency of the detection and ensuring that information about the wear particles in the lubricating oil is captured on time, which improves accuracy and efficiency of detection of the wear condition of the mechanical equipment.

In some embodiments, the detection frequency correlates to flow data of the lubricating oil.

The flow data of the lubricating oil refers to a volume or mass per unit time of the lubricating oil that passes through a cross-section of the lubricating oil wear particle monitoring system. In some embodiments, the flow data of the lubricating oil may be obtained by a flow sensor.

In some embodiments, the detection frequency is positively correlated with the flow data of the lubricating oil. For example, the greater the flow data of the lubricating oil, the higher the detection frequency.

In some embodiments of the present disclosure, dynamic adjustment of the detection frequency can be realized by correlating the detection frequency with the flow data of the lubricating oil. When the flow of the lubricating oil is increased, due to the increase in the count of wear particles that may be contained in the oil and acceleration of the flow, data may be acquired more accurately by increasing the detection frequency to ensure that monitoring results are accurate and timely, thereby improving reliability of the extraction of the wear particle feature signal. The adjustment mechanism helps to optimize detection processes under different flows and improve adaptability and effectiveness of the monitoring system.

In some embodiments, operation S4 further includes determining the adaptive threshold by a threshold determination model based on a curvature distribution feature, the flow data of the lubricating oil, a state feature of the lubricating oil, and features of operating conditions of the equipment. More descriptions regarding the adaptive threshold may be found in the previous descriptions (e.g., FIG. 6-FIG. 7 and related descriptions thereof).

The curvature distribution feature refers to a feature exhibited by a curve obtained by curve fitting the empirical cumulative distribution. For example, the curvature distribution feature may include a location of the maximum of the curvature and a shape feature of the curvature.

In some embodiments, the processor may obtain the curvature distribution feature based on the empirical cumulative distribution of the normalized segmentation entropy by a curve fitting algorithm. For example, the processor may determine the empirical cumulative distribution of the normalized segmentation entropy, fit the empirical cumulative distribution using the curve fitting algorithm to obtain a fitted curve, and finally, extract the location of the maximum of the curvature and the shape feature of the curvature from the fitted curve to form the curvature distribution feature.

The state feature of the lubricating oil refers to a parameter that reflects the performance and condition of the lubricating oil. For example, the state feature of the lubricating oil may include, but is not limited to, a viscosity and a moisture content of the lubricating oil.

In some embodiments, the state features of the lubricating oil may be obtained by a sensor. The sensor may be a capillary viscometer, a capacitive moisture sensor, etc.

The features of operating conditions of the equipment refer to parameters that characterize the operating state of the mechanical equipment. For example, the features of operating conditions of the equipment may include, but are not limited to, a bearing temperature, a vibration amplitude, and a load current of the mechanical equipment.

In some embodiments, the features of operating conditions of the equipment may be obtained by a sensor. The sensor may include, but is not limited to, a temperature sensor, a vibration sensor, a current transformer, etc.

The threshold determination model refers to a model for determining the adaptive threshold. In some embodiments, the threshold determination model is a machine learning model. For example, the threshold determination model may include one or more of a convolutional neural network (CNN) model or other customized models.

FIG. 10 is a schematic diagram illustrating an exemplary threshold determination model according to some embodiments of the present disclosure.

As shown in FIG. 10, inputs to the threshold determination model may include the curvature distribution feature, the flow data of the lubricating oil, the state feature of the lubricating oil, and the characteristics of operating conditions of the equipment, and outputs of the threshold determination model may include the adaptive threshold.

In some embodiments, the threshold determination model may be obtained by training a plurality of training samples with training labels. For example, the plurality of training samples with the training labels may be input into an initial threshold determination model, a loss function is constructed based on the training labels and outputs of the initial threshold determination model, and parameters of the initial threshold determination model may be updated iteratively based on a loss function via gradient descent algorithm or other ways. When a preset condition is satisfied, the training of the model is completed, and a trained threshold determination model is obtained. The preset condition may be that the loss function converges, a count of iterations reaches a threshold, etc.

In some embodiments, the training samples may include a sample curvature distribution feature, sample flow data of sample lubricating oil, a sample state feature of the sample lubricating oil, and sample features of operating conditions of the equipment. The training labels may include preferred adaptive thresholds corresponding to the training samples.

In some embodiments, the training samples may be obtained based on historical data. The processor may take a historical adaptive threshold that minimizes omission rate as the preferred adaptive threshold, i.e., the training label, during a preset subsequent period corresponding to a historical moment in a plurality of extractions of historical signal corresponding to the training samples. The preset subsequent period may be set manually. The omission rate refers to a ratio of a count of omitted wear particles to a total count of wear particles. In some embodiments, the omission rate may be obtained by statistics.

In some embodiments, the threshold determination model includes at least 800 or more multiplication operations in a single execution. In some embodiments, the threshold determination model is at least partially performed by a graphics processing unit (GPU).

In some embodiments of the present disclosure, precise regulation of signal segmentation may be realized by integrally considering the curvature distribution feature, the flow data of the lubricating oil, the state feature of the lubricating oil, and the features of operating conditions of the equipment and using a machine learning model to determine the adaptive threshold. The way of multi-features fusion for determining the adaptive threshold may dynamically adapt thresholds to satisfy different operating conditions, improve accuracy and reliability of the extraction of the wear particle feature signal, and retain effective signals and eliminate the noise interference.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

What is claimed is:

1. A method for extracting a wear particle feature signal based on segmentation entropy, comprising:
S1, obtaining a raw signal to be processed by performing real-time data acquisition of lubricating oil containing ferromagnetic wear particles using a lubricating oil wear particle monitoring system constructed based on an inductive particle detection sensor;
S2, obtaining a preprocessed signal by performing low-pass filtering and harmonic interference suppression on the raw signal to be processed;
S3, dividing the preprocessed signal into a plurality of time domain sequence segments with a sliding window with a fixed length and window shift, calculating segmentation entropy corresponding to each time domain sequence segment among the plurality of time domain sequence segments, and normalizing a segmentation entropy set to obtain normalized segmentation entropy;
S4, obtaining an empirical cumulative distribution of the normalized segmentation entropy, obtaining an adaptive threshold through curve fitting based on the empirical cumulative distribution, and obtaining a plurality of non-zero discrete time domain signal segments by segmenting the preprocessed signal by the adaptive threshold; and
S5, calculating a target signal feature recognition index of each non-zero discrete time domain signal segment among the plurality of non-zero discrete time domain signal segments and setting an index threshold, excluding the non-zero discrete time domain signal segment as residual noise interference when the target signal feature recognition index of the non-zero discrete time domain signal segment is less than the index threshold; and retaining the non-zero discrete time domain signal segment when the target signal feature recognition index of the non-zero discrete time domain signal segment is not less than the index threshold to obtain a final extraction result of the wear particle feature signal.

2. The method of claim 1, wherein in S2, a low-pass filter is configured to perform the low-pass filtering on the raw signal to be processed, and a cutoff frequency f_{c} of the low-pass filter satisfies f_{c}≥2.5f_{d}, and f_{d} is a center frequency of a wear particle induced voltage signal.

3. The method of claim 1, wherein in S2, the harmonic interference suppression is achieved by constructing harmonic components with opposite amplitudes but the same frequencies and phases to be superimposed with the raw signal to be processed after the low-pass filtering, the frequencies are obtained by an iterative interpolation discrete Fourier transform algorithm, and the amplitudes and the phases are obtained using a frequency domain compensation manner.

4. The method of claim 1, wherein S3 includes:
S31, dividing the preprocessed signal using the sliding window with a fixed window length N and window shift Nₘ to obtain J-1 time domain sequence segments;
S32, calculating segmentation entropy of each time domain sequence segment by an equation:
$${ζ}_{j} = N \times ln {S}_{N}$$
wherein ζⱼ denotes segmentation entropy of j-th time domain sequence segment, and S_{N} denotes a sample variance of the j-th time domain sequence segment; and
S33, normalizing segmentation entropy corresponding to the J-1 time domain sequence segments to obtain the normalized segmentation entropy by an equation:
$$\overline{ζ} = \frac{\hat{ζ}}{{‖ \hat{ζ} ‖}_{∞}}$$
wherein \hat{ζ} denotes a decentralized segmentation entropy set ζ={ζ₁, . . . , ζⱼ, . . . , ζ_{J-1}}, and ∥·∥∞ denotes an infinity norm; and elements in the normalized segmentation entropy ζ̅ take values within a range of [−1,1].

5. The method of claim 1, wherein in S4, calculating the adaptive threshold and segmenting the preprocessed signal includes:
S41, delimiting an interval [−1,1] by a fixed step size μ, determining a random variable set X=[−1, −1+μ, . . . , 1]; counting a sample point set R(X)=[R₋₁, R_{-1+μ}, . . . , R₁] in the normalized segmentation entropy which is smaller than a random variable xₘ (m=1, 2, . . . , 2/μ) one by one; determining an empirical cumulative distribution R̅(X) of the normalized segmentation entropy using a normalization equation;
S42, performing curve fitting using a Sigmoid function with a change trend similar to that of the empirical cumulative distribution R̅(X), an expression equation of the Sigmoid function is:
$$S ( t ) = \frac{1}{1 + {e}^{- c t}}$$
wherein c∈R+ denotes an adaptive tuning parameter; an independent variable t takes random variable set X as an input;
S43, calculating a curvature of the Sigmoid function by an equation ρ(t)=|S″(t)|/[(1+S′(t))]^{3/2}, wherein S′(t) and S″(t) denote a first order derivative and a second order derivative of S(t), respectively; inputting a random variable X as an independent variable to obtain a curvature ρ(X); establishing a mapping relationship with the random variable X as a horizontal coordinate and the curvature ρ(X) as a vertical coordinate, and designating a horizontal coordinate random variable x_{ρmax} corresponding to a maximum of the curvature close to 1 as the adaptive threshold; and
S44, setting a preprocessed signal segment corresponding to a variable in the normalized segmentation entropy that is below the adaptive threshold x_{ρmax} to zero and retaining a preprocessed signal segment corresponding to a variable that is above the adaptive threshold x_{ρmax} to obtain H non-zero discrete time domain signal segments.

6. The method of claim 1, wherein in S5, for a non-zero discrete time domain signal segment Δ={Δ₀, Δ₁, . . . , Δ_{L-1}}, the target signal feature recognition index includes:
A. a time domain order index:
$$δ = \{ \begin{matrix}1 , \\ 0 ,\end{matrix} \begin{matrix}Ω > 0 \\ Ω < 0\end{matrix}$$
wherein Ω=Lₘ−Lₙ; Lₘ and Lₙ denote a position of a horizontal coordinate corresponding to a maximum value of a signal amplitude and a position of a horizontal coordinate corresponding to a minimum value of the signal amplitude in the non-zero discrete time domain signal segment, respectively; δ=1 denotes that the non-zero discrete time domain signal segment conforms to a morphology feature of the wear particle induced voltage signal; δ=0 denotes that the non-zero discrete time domain signal segment is directly excluded as a non-wear particle induced voltage signal;
B. a marginal characterization index:
$$ϛ = Q ( \frac{{L}_{n}}{Ω} ) \times Q ( \frac{L - {L}_{m}}{Ω} )$$
wherein Q(·) denotes a function on ε defined by:
$$Q ( ε ) = \{ \begin{matrix}1 , & ε \geq 1 \\ ε , & ε < 1\end{matrix}$$
ζ≈1 denotes that there is a higher probability that the non-zero discrete time domain signal segment has a marginal feature of the wear particle induced voltage signal; ζ<<1 denotes that the non-zero discrete time domain signal segment is classified as the non-wear particle induced voltage signal; and
C. an unbiasedness index:
$$γ = 1 - \frac{\vert {\sum }_{i = {L}_{n}}^{{L}_{m}} {Δ}_{i} \vert}{{\sum }_{i = {L}_{n}}^{{L}_{m}} \vert {Δ}_{i} \vert}$$
wherein Δᵢ denotes i-th element in the non-zero discrete time domain signal segment Δ, γ takes a value within a range of [0,1], and a lower value of γ indicates a higher bias of the non-zero discrete time domain signal segment and a higher probability of the non-zero discrete time domain signal segment being a non-wear particle signal.
